(19) United States
(12) Patent Application Publication
(10) Pub. No.: US 2011/0259583 A1
(43) Pub. Date: Oct. 27, 2011

(54) PROCESS FOR EXTRACTING MINERAL OIL USING SURFACTANTS, ESPECIALLY BASED ON C35 SECONDARY ALCOHOL-CONTAINING ALKYL ALKOXYLATES

(75) Inventors: Christian Bittner, Bensheim (DE); Günter Oetter, Frankenthal (DE); Jack Tinsley, Mannheim (DE); Christian Spindler, Ludwigshafen (DE); Gabriela Alvarez Jürgenson, Mannheim (DE); Sophie Vogel, Mannheim (DE); Petra Neumann, Bohl-Iggelheim (DE); Veronika Wloka, Mannheim (DE); Martin Bock, Hannover (DE)

(73) Assignee: BASF SE, Ludwigshafen (DE)

(21) Appl. No.: 13/093,356

(22) Filed: Apr. 25, 2011

Related U.S. Application Data
(60) Provisional application No. 61/327,124, filed on Apr. 23, 2010.

(30) Foreign Application Priority Data
Apr. 23, 2010 (EP) 10160896.6

(51) Int. Cl. E21B 43/22; C09K 8/584; C07C 305/00

(52) U.S. Cl. 166/270.1; 558/31; 507/254

(57) ABSTRACT

The present invention relates to a process for mineral oil extraction by means of Winsor Type III microemulsion flooding, in which an aqueous surfactant formulation comprising at least one ionic surfactant of the general formula (R¹)(R²)—CH—O-(D)ₙ-(B)ₘ-(A)ₗ-XY^{a-} a/b M^{b+} is injected through injection boreholes into a mineral oil deposit, and crude oil is withdrawn from the deposit through production boreholes. The invention further relates to ionic surfactants of the general formula, to surfactant formulations, and to a process for preparation thereof.

The present invention relates to a process for mineral oil production by means of Winsor type III microemulsion flooding, in which an aqueous surfactant formulation comprising at least one ionic surfactant of the general formula

(R¹)(R²)—CH—O-(D)ₙ-(B)ₘ-(A)ₗ-XY^{a-}a/bM^{b+} (I)

is injected through injection boreholes into a mineral oil deposit, and crude oil is withdrawn from the deposit through production boreholes. The invention further relates to ionic surfactants of the general formula, to surfactant formulations and to processes for preparation thereof.

In natural mineral oil deposits, mineral oil is present in the cavities of porous reservoir rocks which are sealed toward the surface of the earth by impervious top layers. The cavities may be very fine cavities, capillaries, pores or the like. Fine pore necks may, for example, have a diameter of only about 1 vim. As well as mineral oil, including fractions of natural gas, a deposit comprises water with a greater or lesser salt content.

In mineral oil production, a distinction is generally drawn between primary, secondary and tertiary production. In primary production, the mineral oil flows, after commencement of drilling of the deposit, of its own accord through the borehole to the surface owing to the autogenous pressure of the deposit.

After primary production, secondary production is therefore used. In secondary production, in addition to the boreholes which serve for the production of the mineral oil, the so-called production bores, further boreholes are drilled into the mineral oil-bearing formation. Water is injected into the deposit through these so-called injection bores in order to maintain the pressure or to increase it again. As a result of the injection of the water, the mineral oil is forced slowly through the cavities into the formation, proceeding from the injection bore in the direction of the production bore. However, this only works for as long as the cavities are completely filled with oil and the more viscous oil is pushed onward by the water. As soon as the mobile water breaks through cavities, it flows on the path of least resistance from this time, i.e. through the channel formed, and no longer pushes the oil onward.

By means of primary and secondary production, generally only approx. 30 to 35% of the amount of mineral oil present in the deposit can be produced.

It is known that the mineral oil yield can be enhanced further by measures for tertiary oil production. A review of tertiary oil production can be found, for example, in “Journal of Petroleum Science of Engineering 19 (1998)”, pages 265 to 280. Tertiary oil production includes, for example, thermal methods in which hot water or steam is injected into the deposit. This lowers the viscosity of the oil. The flow medium used may likewise be gases such as CO₂ or nitrogen.

Tertiary mineral oil production also includes methods in which suitable chemicals are used as assistants for oil production. These can be used to influence the situation toward the end of the water flow and as a result also to produce mineral oil hitherto held firmly within the rock formation.

Viscous and capillary forces act on the mineral oil which is trapped in the pores of the deposit rock toward the end of the secondary production, the ratio of these two forces relative to one another being determined by the microscopic oil separation. By means of a dimensionless parameter, the so-called capillary number, the action of these forces is described. It is the ratio of the viscosity forces (velocity×viscosity of the forcing phase) to the capillary forces (interfacial tension between oil and water×wetting of the rock):

$${N}_{c} = \frac{μ v}{σ cos θ} .$$

In this formula, μ is the viscosity of the fluid mobilizing mineral oil, ν is the Darcy velocity (flow per unit area), σ is the interfacial tension between liquid mobilizing mineral oil and mineral oil, and θ is the contact angle between mineral oil and the rock (C. Melrose, C. F. Brandner, J. Canadian Petr. Techn. 58, October-December, 1974). The higher the capillary number, the greater the mobilization of the oil and hence also the degree of oil removal.

It is known that the capillary number toward the end of secondary mineral oil production is in the region of about 10⁻⁶ and that it is necessary to increase the capillary number to about 10⁻³ to 10⁻² in order to be able to mobilize additional mineral oil.

For this purpose, it is possible to conduct a particular form of the flooding method—what is known as Winsor type III microemulsion flooding. In Winsor type III microemulsion flooding, the injected surfactants should form a Winsor type III microemulsion with the water phase and oil phase present in the deposit. A Winsor type III microemulsion is not an emulsion with particularly small droplets, but rather a thermodynamically stable, liquid mixture of water, oil and surfactants. The three advantages thereof are that
a very low interfacial tension σ between mineral oil and aqueous phase is thus achieved,
it generally has a very low viscosity and as a result is not trapped in a porous matrix,
it forms with even the smallest energy inputs and can remain stable over an infinitely long period (conventional emulsions, in contrast, require high shear forces which predominantly do not occur in the reservoir, and are merely kinetically stabilized).

The Winsor type III microemulsion is in an equilibrium with excess water and excess oil. Under these conditions of microemulsion formation, the surfactants cover the oil-water interface and lower the interfacial tension a more preferably to values of <10⁻² mN/m (ultra-low interfacial tension). In order to achieve an optimal result, the proportion of the microemulsion in the water-microemulsion-oil system, with a defined amount of surfactant, should by its nature be at a maximum, since this allows lower interfacial tensions to be achieved.

In this manner, it is possible to alter the form of the oil droplets (interfacial tension between oil and water is lowered to such a degree that the smallest interface state is no longer favored and the spherical form is no longer preferred), and they can be forced through the capillary openings by the flooding water.

When all oil-water interfaces are covered with surfactant, in the presence of an excess amount of surfactant, the Winsor type III microemulsion forms. It thus constitutes a reservoir for surfactants which cause a very low interfacial tension between oil phase and water phase. By virtue of the Winsor type III microemulsion being of low viscosity, it also migrates through the porous deposit rock in the flooding process (emulsions, in contrast, can become trapped in the porous matrix and block deposits). When the Winsor type III microemulsion meets an oil-water interface as yet uncovered with surfactant, the surfactant from the microemulsion can significantly lower the interfacial tension of this new interface, and lead to mobilization of the oil (for example by deformation of the oil droplets).

The oil droplets can subsequently combine to a continuous oil bank. This has two advantages:

Firstly, as the continuous oil bank advances through new porous rock, the oil droplets present there can coalesce with the bank.

Moreover, the combination of the oil droplets to give an oil bank significantly reduces the oil-water interface and hence surfactant no longer required is released again. Thereafter, the surfactant released, as described above, can mobilize oil droplets remaining in the formation.

Winsor type III microemulsion flooding is consequently an exceptionally efficient process, and requires much less surfactant compared to an emulsion flooding process. In microemulsion flooding, the surfactants are typically optionally injected together with co-solvents and/or basic salts (optionally in the presence of chelating agents). Subsequently, a solution of thickened polymer is injected for mobility control. A further variant is the injection of a mixture of thickening polymer and surfactants, co-solvents and/or basic salts (optionally with chelating agent), and then a solution of thickening polymer for mobility control. These solutions should generally be clear in order to prevent blockages of the reservoir.

The requirements on surfactants for tertiary mineral oil production differ significantly from requirements on surfactants for other applications: suitable surfactants for tertiary oil production should reduce the interfacial tension between water and oil (typically approx. 20 mN/m) to particularly low values of less than 10⁻² mN/m in order to enable sufficient mobilization of the mineral oil. This has to be done at the customary deposit temperatures of approx. 15° C. to 130° C. and in the presence of water of high salt contents, more particularly also in the presence of high proportions of calcium and/or magnesium ions; the surfactants thus also have to be soluble in deposit water with a high salt content.

To fulfill these requirements, there have already been frequent proposals of mixtures of surfactants, especially mixtures of anionic and nonionic surfactants.

U.S. Pat. No. 3,391,750 discloses a surfactant mixture comprising alkyl alkoxy sulfates of the C₁₁-C₁₅—2 to 6 EO—sulfate type. The alkyl radical comprises secondary radicals. Use as a foaming agent for removal of water in compressed air drilling is described.

U.S. Pat. No. 3,500,923 discloses the use of surfactant solutions comprising alkyl propoxy sulfates for enhancement of oil production with the aid of surfactant flooding. These surfactants can be combined as disclosed with alkyl ethoxy sulfates based on secondary alcohols. An example specified was Tergitol products from Union Carbide.

Journal of Colloid and Interface Science 1986, 114 (2), 342-356 describes monoisomeric alkyl ethoxy sulfonates for low interfacial tensions in a salt-rich environment. These are based on secondary alcohols having 16 or 18 carbon atoms. The alcohol is in the 2 position.

WO 2008/079855 A1 describes surfactants for mineral oil deposit exploitation. These surfactants are said be based on secondary alcohols. The text mentions a chain length of C10 to C24. No statement as to the position of the alcohol was given. These alcohols are prepared via the oxidation of paraffins or the reaction of olefins with ethylene glycol.

In WO 2009/058654 A1 is a preparation process for surfactants based on secondary alcohols having 9 to 30 carbon atoms described. The alcohol group is usually in the 2 or 3 position. These alcohols are obtained by oxidation of paraffins with oxygen or reaction of paraffins with orthoboric acid. This is followed by alkoxylation with DMC catalysis and then a sulfation.

The use parameters, for example type, concentration and mixing ratio of the surfactants used with respect to one another, are therefore adjusted by the person skilled in the art according to the conditions existing in a given oil formation (for example temperature and salt content).

As described above, mineral oil production is proportional to the capillary number. The lower the interfacial tension between oil and water, the higher it is. The higher the mean number of carbon atoms in the crude oil, the more difficult it is to achieve low interfacial tension. Suitable surfactants for low interfacial tensions are those which possess a long alkyl radical. The longer the alkyl radical, the better it is possible to reduce the interfacial tensions. However, the availability of such compounds is very limited.

It is therefore an object of the invention to provide a particularly efficient surfactant for use for surfactant flooding, and an improved process for tertiary mineral oil production. It is a further object of the invention to provide a process for preparing this surfactant.

Accordingly, a surfactant, and a process are provided for tertiary mineral oil production by means of Winsor type III microemulsion flooding, in which an aqueous surfactant formulation comprising at least one surfactant is injected through at least one injection borehole into a mineral oil deposit, the interfacial tension between oil and water is lowered to values of <0.1 mN/m, preferably to values of <0.05 mN/m, more preferably to values of <0.01 mN/m, and crude oil is withdrawn from the deposit through at least one production borehole, wherein the surfactant formulation comprises at least one surfactant of the general formula

(R¹)(R²)—CH—O-(D)ₙ-(B)ₘ-(A)ₗ-XY^{a-}a/bM^{b+}, where
R¹ is a linear or branched, saturated or unsaturated, aliphatic hydrocarbon radical having 7 to 21 carbon atoms,
R² is a linear or branched, saturated or unsaturated, aliphatic hydrocarbon radical having 7 to 21 carbon atoms, and
R¹ is either identical to R² or has a maximum of four more carbon atoms than R²,
A is ethyleneoxy,
B is propyleneoxy, and
D is butyleneoxy,
l is from 0 to 99,
m is from 0 to 99 and
n is from 0 to 99,
X is an alkyl or alkylene group having 0 to 10 carbon atoms,
M^{b+} is a cation,
Y^{a-} is selected from the group of sulfate groups, sulfonate groups, carboxylate groups and phosphate groups,
b is 1, 2 or 3,
a is 1 or 2, where
   the A, B and D groups may be distributed randomly, alternatingly, or in the form of two, three, four or more blocks in any sequence, the sum of l+m+n is in the range from 0 to 99 and the proportion of 1,2-butylene oxide, based on the total amount of butylene oxide, is at least 80%.

In a preferred embodiment of the invention,
R¹ is a linear, saturated or unsaturated, aliphatic hydrocarbon radical having 15 to 17 carbon atoms,
R² is a linear, saturated or unsaturated, aliphatic hydrocarbon radical having 15 to 17 carbon atoms.

In a particularly preferred embodiment, R¹ is identical to R² and is a linear, saturated aliphatic hydrocarbon radical having 17 carbon atoms.

Additionally provided has been a surfactant mixture for mineral oil production, which comprises at least one surfactant of the general formula defined above.

With regard to the invention, the following should be stated specifically:

In the above-described process according to the invention for mineral oil production by means of Winsor type III microemulsion flooding, an aqueous surfactant formulation comprising at least one surfactant of the general formula is used. It may additionally comprise further surfactants and/or other components.

In the process according to the invention for tertiary mineral oil production by means of Winsor type III microemulsion flooding, the use of the inventive surfactant lowers the interfacial tension between oil and water to values of <0.1 mN/m, preferably to <0.05 mN/m, more preferably to <0.01 mN/m. The interfacial tension between oil and water is thus lowered to values in the range from 0.1 mN/m to 0.0001 mN/m, preferably to values in the range from 0.05 mN/m to 0.0001 mN/m, more preferably to values in the range from 0.01 mN/m to 0.0001 mN/m.

The at least one surfactant can be encompassed by the general formula (R¹)(R²)—CH—O-(D)ₙ-(B)ₘ-(A)ₗ-XY^{a-} a/b M^{b+}. As a result of the preparation, it is also possible for a plurality of different surfactants of the general formula to be present in the surfactant formulation.

The R¹ radical is a linear or branched, saturated or unsaturated, aliphatic hydrocarbon radical having 7 to 21 carbon atoms. The R² radical is a linear or branched, saturated or unsaturated aliphatic hydrocarbon radical having 7 to 21 carbon atoms. R¹ is either identical to R² or has a maximum of four carbon atoms more than R².

In a preferred embodiment R¹ is a linear, saturated or unsaturated, aliphatic hydrocarbon radical having 7 to 17 carbon atoms. The R² radical is preferably a linear, saturated or unsaturated, aliphatic hydrocarbon radical having 7 to 17 carbon atoms.

The R¹ radical is more preferably a linear or branched, saturated or unsaturated, aliphatic hydrocarbon radical having 15 to 17 carbon atoms. The R² radical is preferably a linear or branched, saturated or unsaturated, aliphatic hydrocarbon radical having 15 to 17 carbon atoms. R¹ is preferably either identical to R² or has a maximum of two carbon atoms more than R².

The R¹ radical is more preferably a linear saturated or unsaturated aliphatic hydrocarbon radical having 15 to 17 carbon atoms. The R² radical is more preferably a linear or branched, saturated or unsaturated, aliphatic hydrocarbon radical having 15 to 17 carbon atoms. R¹ is more preferably identical to R².

In a particularly preferred embodiment, R¹ is identical to R² and is a linear saturated aliphatic hydrocarbon radical having 17 carbon atoms.

In a further preferred embodiment of the invention, the R¹ or R² radical is an aliphatic linear saturated C₁₅H₃₁ radical or aliphatic linear saturated C₁₇H₃₅ radical or aliphatic linear unsaturated C₁₇ radical having 0.1 to 3 double bonds. In a further preferred embodiment of the invention, the R¹ or R² radical is an aliphatic branched saturated C₁₅H₃₁ radical or an aliphatic branched saturated C₁₆H₃₃ radical. Particular preference is given to a degree of branching in R¹ or R² in the range of 0.1-5 and most preferably of 0.1-1.5.

The term “degree of branching” is defined here in a manner known in principle as the number of methyl groups in a molecule of the alcohol minus 1. The mean degree of branching is the statistical mean of the degrees of branching of all molecules in a sample.

In a preferred embodiment of the invention, n and m have a value of n=2 to 10 and m=5 to 9. It is preferred here in each case that D is 1,2-butylene oxide to an extent of more than 80%, and that the alkylene oxides, beginning at the alcohol, have the sequence D-B-A. The alkylene oxides are arranged in blocks to an extent of more than 90%. In a particularly preferred embodiment, n and m have values of n=0 and m=5 to 9.

(R¹)(R²)—CH is preferably a branched aliphatic, saturated or unsaturated hydrocarbon radical having 15 to 43 carbon atoms. In a further preferred embodiment it is a branched aliphatic, saturated or unsaturated hydrocarbon radical having 15 to 35 carbon atoms. However, it is more preferably a branched aliphatic, saturated or unsaturated hydrocarbon radical having 31 to 35 carbon atoms. However, it is most preferably a branched aliphatic, saturated or unsaturated hydrocarbon radical having 35 carbon atoms.

A branched aliphatic hydrocarbon radical (R¹)(R²)—CH generally has a degree of branching of 1 to 11, preferably 1 to 7, more preferably 1.

In the above formula, A means ethyleneoxy. B means propyleneoxy and D means butyleneoxy.

In the above-defined general formula I, m and n are each integers. It is, however, clear to the person skilled in the art in the field of polyalkoxylates that this definition is the definition of a single surfactant in each case. In the case of presence of surfactant mixtures or surfactant formulations which comprise a plurality of surfactants of the general formula, the numbers l, m and n are each mean values over all molecules of the surfactants, since the alkoxylation of alcohol with ethylene oxide and/or propylene oxide and/or butylene oxide in each case affords a certain distribution of chain lengths. This distribution can be described in a manner known in principle by the polydispersity D. D=M_{w}/Mₙ is the quotient of the weight-average molar mass and the number-average molar mass. The polydispersity can be determined by means of the methods known to those skilled in the art, for example by means of gel permeation chromatography.

In the above general formula I is from 0 to 99, preferably 1 to 40, more preferably 1 to 20.

In the above general formula III is from 0 to 99, preferably 1 to 20, more preferably 5 to 9.

In the above general formula n is from 0 to 99, preferably 2 to 30, more preferably 2 to 10. In a further preferred embodiment, n is 0.

According to the invention, the sum of l+m+n is a number in the range from 3 to 99, preferably in the range from 5 to 50, more preferably in the range from 8 to 39.

In the prior art, there is often no specific information with regard to the description of C₄ epoxides. This may generally be understood to mean 1,2-butylene oxide, 2,3-butylene oxide, isobutylene oxide, and mixtures of these compounds. The composition is generally dependent on the C₄ olefin used, and to a certain degree on the oxidation process.

According to the present invention the proportion of 1,2-butyleneoxy, based on the total amount of butyleneoxy (D), is at least 80%, preferably at least 85%, preferably at least 90%, more preferably at least 95%, of 1,2-butyleneoxy.

The ethyleneoxy (A), propyleneoxy (B) and butyleneoxy (D) group(s) are randomly distributed, alternatingly distributed, or are in the form of two, three, four, five or more blocks in any sequence.

In a preferred embodiment of the invention, in the presence of a plurality of different alkyleneoxy blocks, the sequence (R¹)(R²)—CH, butyleneoxy block, propyleneoxy block, ethyleneoxy block is preferred. The butylene oxide used should comprise 80% of 1,2-butylene oxide, preferably >90% of 1,2-butylene oxide.

In the above general formula, X is an alkylene group or alkenylene group having 0 to 10, preferably 0 to 3 carbon atoms. The alkylene group is preferably a methylene, ethylene or propylene group. X is more preferably a single bond.

In the above general formula Y^{a-} is a sulfonate, sulfate or carboxyl group or phosphate group. Y is preferably a sulfonate, sulfate or carboxyl group. Thus, a may be 1 or 2.

In the above formula M^{b+} is a cation, preferably a cation selected from the group of Na⁺, K⁺, Li⁺, NH₄⁺, H⁺, Mg²⁺ and Ca²⁺. M⁺ is more preferably a cation selected from the group of Na⁺, K⁺ or NH₄⁺. Overall, b may have values of 1, 2 or 3.

The present invention thus further relates to a process for preparing the surfactant or the surfactants of the general formula I, as defined above.

Thus, in accordance with the invention, a process is provided for preparing surfactants of the general formula (I)

(R¹)(R²)—CH—O-(D)ₙ-(B)ₘ-(A)ₗ-XY^{a-}a/bM^{b+} (I)

in which R¹, R², A, B, D, l, m, n, X, a, b, M^{b+} and Y^{a-} are each as defined above.

The process comprises the steps of
(a) preparation of ketones of the general formula (II)

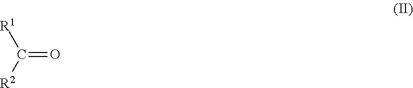

in which R¹ and R², each of which is as defined above, by reaction of two carboxylic acids of the general formulae (IIa/IIb), R¹—COOH(IIa) and R²—COOH (IIb),
(b) reduction/hydrogenation of the ketone of the general formula (II) prepared in process step (a) to give the secondary alcohol of the formula R¹(R²)—CHOH,
(c) alkoxylation of the secondary alcohols of the formula R¹(R²)—CHOH obtained in process step (b), and
(d) introduction of the anionic group XY⁻.

The preparation of the ketone or of the ketones in process step (a) is generally accomplished by conversion of mixtures of (IIa) and (IIb) in the gas phase in the presence of a catalyst, the catalysts used being those with an active material consisting of at least 50% by weight of titanium dioxide with a specific surface area greater than 10 m²/g. The content of titanium dioxide with the surface area mentioned in the active material of the catalysts is 50 to 100% by weight, preferably 50 to 99.95% by weight.

If R¹ is the same as R², the ketone of the general formula II is a symmetric ketone; in an unsymmetric ketone, R¹ and R², in contrast, are different.

The selectivity for the desired ketone is particularly high when the specific surface area of the catalyst is greater than 10 m²/g, preferably 20 to 200 m²/g, and when the catalyst comprises 0.05 to 50% by weight, preferably 1 to 10% by weight, of at least one metal oxide selected from the first or second main group of the periodic table, especially from the elements lithium, sodium, potassium, or from the group of the rare earth metals, especially from the elements lanthanum or cerium, or mixtures of these oxides. In a particular embodiment, the catalyst comprises 0.05 to 50% by weight of lithium oxide, sodium oxide or potassium oxide.

The titanium dioxide is advantageously used in the form of anatase.

The catalysts may be used in the form of impregnated or mixed catalysts.

Preparation of impregnated catalysts: The starting material used is high-surface area titanium dioxide, for example pyrogenic TiO₂ or dried metatitanic acid, which is converted to a shapable state with addition of peptidizing agents in a kneader or mixer. The kneaded mixture is extruded, dried and calcined. After determining the porosity, an impregnation solution whose volume corresponds to the filling of the support is used. The impregnation is performed by adding the impregnation solution, advantageously by spray application, to the initially charged support in a rotating drum. Suitable for preparation of the impregnation solutions are all soluble salts which decompose to oxides with no further residues on calcination.

Preparation of mixed catalysts: the mixed catalysts are prepared in a similar manner to the supports of the supported catalysts. In addition, the corresponding salt solutions are added to the TiO₂ kneading material in the kneader, and good mixing is ensured. Shaping, drying and calcination are effected as in the support production.

The catalysts, as described above, can be regenerated again by thermal treatment with air or with air/nitrogen mixtures at 450 to 550° C.

In the process according to process step (a), decarboxylation and dehydration (elimination of CO₂ and water) give ketones or ketone mixtures IIA, IIB and IIC:

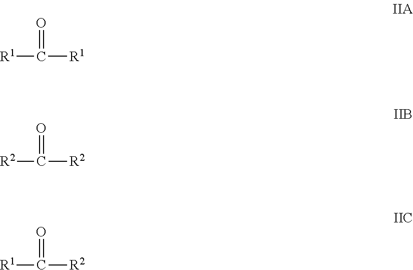

In the case of preparation of mixed ketones IIC, the statistical ratio of IIA:IIB:IIC=0.25:0.25:0.5 may be attained owing to chemical similarity. Owing to the different activities of the starting acids, the active acid preferably reacts with itself, such that it is available for the formation of the mixed ketone only in a reduced mass. It is a significant advantage of the catalysts described that the less reactive acid is also activated more strongly than in the case of the conventional catalysts, such that the activation differences are reduced. The yield of mixed ketone can be increased by using the more active acid up to a 10 molar excess relative to the less active acid, but correspondingly large amounts of the symmetric ketone of the excess acid are obtained in this case. In the case of preparation of unsymmetric ketones IIC, instead of the carboxylic acid R²COOH, it is likewise possible to use the symmetric ketone R²—CO—R² formed in the reaction therefrom as the reactant, which then likewise reacts according to the following equation to give the desired ketone IIC:

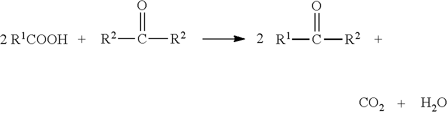

The dehydrating decarboxylation reaction is undertaken preferably at standard pressure and at temperatures of 300 to 600° C., especially at 350 to 450° C., by passing the acid vapors preheated to this temperature through a fixed bed oven filled with catalyst extrudates, granules, tablets, spall or rings, or by performing the reaction in a fluidized bed oven. In the case of comparatively nonvolatile acids, it may also be advisable to work under reduced pressure. In general, 200 to 500 g/h of the ketones can be prepared per liter of catalyst. Step (a) is preferably effected in the gas phase in the presence of a catalyst at 300-500° C.

After passing through the catalyst zone, the vapors are cooled and worked up as usual. In general, conversions of 97 to 100% are achieved, and ketone yields based thereon of 55 to 85% in the case of unsymmetric ketones and 90 to 99% in the case of symmetric ketones.

It is possible to use carboxylic acids which comprise up to 50% by weight of water; since the water frequently has a favorable effect on the activation time of the catalysts (less carbon separates out on the catalysts), it is even appropriate to admix the carboxylic acids with 1 to 50% by weight of water.

The reduction or hydrogenation of the ketone or ketone mixture which is obtained in process step (a) and is of the general formula (II) to the corresponding alcohol R¹(R²)—CH—OH is generally undertaken in the presence of a heterogeneous copper catalyst, the catalytically active component of the catalyst additionally comprising aluminum and at least one further metal selected from lanthanum, tungsten, molybdenum, titanium, zirconium and mixtures thereof. In process step (b), a heterogeneous catalyst comprising copper, aluminum and at least one further metal selected from lanthanum, tungsten, molybdenum, titanium, zirconium and mixtures thereof is thus used for the hydrogenation.

The heterogeneous hydrogenation catalysts used may be unsupported catalysts or supported catalysts. These may be used in the form of catalysts of homogeneous composition, impregnated catalysts, coated catalysts and precipitated catalysts. Suitable catalysts may comprise the metals in oxidic form, reduced form (elemental form) or a combination thereof. Metals which are stable in more than one oxidation state can be used completely in one of the oxidation states or in different oxidation states.

A specific embodiment of catalysts which are particularly advantageously suitable for use in process step (b) is that of catalysts which comprise copper in oxidic form and optionally additionally in elemental form. The precipitated catalysts useable in step (b) then comprise preferably at least 25% by weight, more preferably at least 35% by weight, of copper in oxidic and/or elemental form, based on the total weight of the catalyst. Particularly preferred catalysts comprise the following metals: copper, aluminum lanthanum or copper, aluminum and tungsten.

A frequently employed process for preparing such catalysts consists in the impregnation of support materials with solutions of the catalyst components, which are then converted to the catalytically active state by thermal treatment, decomposition or reduction.

A further suitable process for preparing catalysts comprises the precipitation of at least one catalyst component. Different catalyst components can be precipitated in succession, or two or more than two catalyst components can be precipitated in coprecipitation. For instance, to prepare a shaped catalyst body, a copper compound, at least one further metal compound and optionally at least one additive can be precipitated and then subjected to drying, calcination and shaping. The precipitation can be performed in the presence of a support material. Suitable starting materials for the precipitation are metal salts and metal complexes. The metal compounds used for the precipitations may in principle be all known metal salts which are soluble in the solvents used for application to the support. These include, for example, nitrates, carbonates, acetates, oxalates or ammonium complexes. In a preferred embodiment, at least one metal nitrate is used. Preference is given to using an aqueous medium for the precipitation.

The hydrogenation in process step (b) is effected preferably at a temperature in the range from 100 to 320° C., more preferably from 150 to 250° C., especially from 150 to 220° C.

The hydrogenation in process step (b) is effected preferably at a pressure within a range from 100 to 325 bar, more preferably from 150 to 300 bar, especially from 150 to 220 bar.

The molar ratio of hydrogen to ketone is preferably 10:1 to 1000:1, more preferably 12.5:1 to 500:1.

The catalyst hourly space velocity in continuous mode is preferably 0.1 to 1 kg and more preferably 0.2 to 0.5 kg of ketone to be hydrogenated/kg (catalyst)×hour. The hydrogenation can be performed either continuously or batchwise. The hydrogenation is preferably performed continuously. The hydrogenation output consists essentially of the alcohols R¹(R²)CHOH.

In general, the hydrogenation in process step (b) can be undertaken in the melt, in solution, in suspension mode or over a fixed bed. The hydrogenation is effected in n reactors connected in series, where n is 1, 2, 3, 4, 5, 6 or 7.

In a preferred embodiment, the hydrogenation is performed in solution. In a particularly preferred embodiment, the hydrogenation is performed in a solution which comprises 1 to 50% by weight of petroleum spirit, ethers such as THF and dioxane, and/or branched and/or unbranched C₃-C₁₈ alcohols.

The alcohols obtained in process step (b) are prepared in a manner known in principle by alkoxylating corresponding alcohols (R¹)(R²)—CH—OH in process step (c). The performance of such alkoxylation is known in principle to those skilled in the art. It is likewise known to those skilled in the art that the molar mass distribution of the alkoxylates can be influenced through the reaction conditions, especially the selection of the catalyst.

The surfactants of the general formula can preferably be prepared in process step (c) by base-catalyzed alkoxylation. In this case, the alcohol (R¹)(R²)—CH—OH can be admixed in a pressure reactor with alkali metal hydroxides, preferably potassium hydroxide, or with alkali metal alkoxides, for example sodium methoxide. Water still present in the mixture can be drawn off by means of reduced pressure (for example <100 mbar) and/or increasing the temperature (30 to 150° C.). Thereafter, the alcohol is present in the form of the corresponding alkoxide. This is followed by intertization with inert gas (for example nitrogen) and stepwise addition of the alkylene oxide(s) at temperatures of 60 to 180° C. up to a maximum pressure of 10 bar. In a preferred embodiment, the alkylene oxide is metered in initially at 130° C. In the course of the reaction, the temperature rises up to 170° C. as a result of the heat of reaction released. In a further preferred embodiment of the invention, the butylene oxide is first added at a temperature in the range from 135 to 145° C., then the propylene oxide is added at a temperature in the range from 130 to 145° C., and then the ethylene oxide is added at a temperature in the range from 125 to 145° C. At the end of the reaction, the catalyst can be centralized, for example, by adding acid (for example acetic acid or phosphoric acid) and filtered off if required.

However, the alkoxylation of the alcohols (R¹)(R²)—CH—OH can also be undertaken by means of other methods, for example by acid-catalyzed alkoxylation. In addition, it is possible to use, for example double hydroxide clays, as described in DE 4325237 A1, or it is possible to use double metal cyanide catalysts (DMC catalysts). Suitable DMC catalysts are disclosed, for example in DE 10243361 A1, especially in paragraphs [0029] to [0041] and the literature cited therein. For example, it is possible to use catalysts of the Zn—Co type. To perform the reaction, the alcohol (R¹)(R²)—CH—OH can be admixed with the catalyst, and the mixture can be dewatered as described above and reacted with the alkylene oxides as described. Typically not more than 1000 ppm of catalyst based on the mixture are used, and the catalyst can remain in the product owing to this small amount. The amount of catalyst may generally be less than 1000 ppm, for example 250 ppm or less.

The anionic group is finally introduced in process step (d). This is known in principle to those skilled in the art. The anionic group XY^{a-} is composed of the functional group Y^{a-}, which is a sulfate, sulfonate, carboxylate or phosphate group, and the spacer X, which in the simplest case may be a single bond (“alkyl or alkylene group having 0 carbon atoms”). In the case of a sulfate group, it is possible, for example, to employ the reaction with sulfuric acid, chlorosulfonic acid or sulfur trioxide in a falling-film reactor with subsequent neutralization. In the case of a sulfonate group it is possible, for example, to employ the reaction with propane sultone and subsequent neutralization, with butane sultone and subsequent neutralization, with vinylsulfonic acid sodium salt, or with 3-chloro-2-hydroxypropanesulfonic acid sodium salt. To prepare sulfonates, the terminal OH group can also be converted to a chloride, for example with phosgene or thionyl chloride, and then, for example, reacted with sulfite. In the case of a carboxylate group, it is possible, for example, to employ the oxidation of the alcohol with oxygen and subsequent neutralization, or the reaction with chloroacetic acid sodium salt. Carboxylates can, for example, also be obtained by Michael addition of (meth)acrylic acid or ester. Phosphates can, for example, be obtained by esterification reaction with phosphoric acid or phosphorus pentachloride.

### Further Surfactants

In addition to the surfactants of the general formula (I), the formulation may additionally optionally comprise further surfactants. These are, for example, anionic surfactants of the alkylarylsulfonate or olefinsulfonate (alpha-olefinsulfonate or internal olefinsulfonate) type and/or nonionic surfactants of the alkyl ethoxylate or alkyl polyglucoside type or betaine surfactants. These further surfactants may especially also be oligomeric or polymeric surfactants. It is advantageous to use such polymeric co-surfactants to reduce the amount of surfactants needed to form a microemulsion. Such polymeric co-surfactants are therefore also referred to as “microemulsion boosters”. Examples of such polymeric surfactants comprise amphiphilic block copolymers which comprise at least one hydrophilic block and at least one hydrophobic block. Examples comprise polypropylene oxide-polyethylene oxide block copolymers, polyisobutene-polyethylene oxide block copolymers, and comb polymers with polyethylene oxide side chains and a hydrophobic main chain, where the main chain preferably comprises essentially olefins or (meth)acrylates as monomers. The term “polyethylene oxide” here should in each case include polyethylene oxide blocks comprising propylene oxide units as defined above. Further details of such surfactants are disclosed in WO 2006/131541 A1.

### Process for Mineral Oil Production

In the process according to the invention for mineral oil production, a suitable aqueous formulation of the surfactants of the general formula is injected through at least one injection borehole into the mineral oil deposit, and crude oil is withdrawn from the deposit through at least one production borehole. The term “crude oil” in this context of course does not mean single-phase oil, but rather the usual crude oil-water emulsions. In general, a deposit is provided with several injection boreholes and with several production boreholes.

The main effect of the surfactant lies in the reduction of the interfacial tension between water and oil—desirably to values significantly <0.1 mN/m. After the injection of the surfactant formulation, known as “surfactant flooding”, or preferably the Winsor type III “microemulsion flooding”, the pressure can be maintained by injecting water into the formation (“water flooding”) or preferably a higher-viscosity aqueous solution of a polymer with strong thickening action (“polymer flooding”). Also known, however, are techniques by which the surfactants are first of all allowed to act on the formation. A further known technique is the injection of a solution of surfactants and thickening polymers, followed by a solution of thickening polymer. The person skilled in the art is aware of details of the industrial performance of “surfactant flooding”, “water flooding”, and “polymer flooding”, and employs an appropriate technique according to the type of deposit.

For the process according to the invention, an aqueous formulation which comprises surfactants of the general formula is used. In addition to water, the formulations may optionally also comprise water-miscible or at least water-dispersible organic substances or other substances. Such additives serve especially to stabilize the surfactant solution during storage or transport to the oil field. The amount of such additional solvents should, however, generally not exceed 50% by weight, preferably 20% by weight. In a particularly advantageous embodiment of the invention, exclusively water is used for formulation. Examples of water-miscible solvents include especially alcohols such as methanol, ethanol and propanol, butanol, sec-butanol, pentanol, butyl ethylene glycol, butyl diethylene glycol or butyl triethylene glycol.

According to the invention, the proportion of the surfactants of the general formula is at least 30% by weight based on the proportion of all surfactants present, i.e. the surfactants of the general formula and optionally present surfactants. The proportion is preferably at least 50% by weight.

The mixture used in accordance with the invention can preferably be used for surfactant flooding of deposits. It is especially suitable for Winsor type III microemulsion flooding (flooding in the Winsor III range or in the range of existence of the bicontinuous microemulsion phase). The technique of microemulsion flooding has already been described in detail at the outset.

In addition to the surfactants, the formulations may also comprise further components, for example C₄- to C₈ alcohols and/or basic salts (so-called “alkali surfactant flooding”). Such additives can be used, for example, to reduce retention in the formation. The ratio of the alcohols based on the total amount of surfactant used is generally at least 1:1—however, it is also possible to use a significant excess of alcohol. The amount of basic salts may typically range from 0.1% by weight to 5% by weight.

The deposits in which the process is employed generally have a temperature of at least 10° C., for example 10 to 150° C., preferably a temperature of at least 15° C. to 120° C. The total concentration of all surfactants together is 0.05 to 5% by weight, based on the total amount of the aqueous surfactant formulation, preferably 0.1 to 2.5% by weight. The person skilled in the art makes a suitable selection according to the desired properties, especially according to the conditions in the mineral oil formation. It is clear here to the person skilled in the art that the concentration of the surfactants can change after injection into the formation because the formulation can mix with formation water, or surfactants can also be absorbed on solid surfaces of the formation. It is the great advantage of the mixture used in accordance with the invention that the surfactants lead to a particularly good lowering of interfacial tension.

It is of course possible and also advisable first to prepare a concentrate which is only diluted on site to the desired concentration for injection into the formation. In general, the total concentration of the surfactants in such a concentrate is 10 to 45% by weight.

The examples which follow are intended to illustrate the invention in detail:

### Part I: Synthesis of the Surfactants

### General Method 1: Preparation of the Ketone

### 1.1 Preparation of Stearone

Stearic acid (125 g/h), water (10 g/h) and nitrogen (20 l/h) were evaporated continuously and passed at 380° C. over 100 g of a catalyst as described in EP352674, Example 1.2. The biphasic reaction output was condensed and the organic phase was removed and analyzed by GC (DB-5 30 m 0.32 mm i.d., 0.1 μm, 100° C.—2 min—6° C./min to 300° C.—60 min, injection volume: 5 μl) and OH number. The conversion according to OH number was 99.5%; the selectivity for stearone was 97%.

### 1.2 Preparation of a Mixture of C31, C33 and C35 Ketones

A mixture of stearic acid and palmitic acid (50:50 w/w, 125 g/h), water (10 g/h) and nitrogen (20 I/h) was evaporated continuously and passed at 380° C. over 100 g of a catalyst as described in EP352674, Example 1.2. The biphasic reaction output was condensed, and the organic phase was removed and analyzed by GC (DB-5 30 m 0.32 mm i.d., 0.1 μm 100° C.—2 min—6° C./min to 300° C.—60 min, injection volume: 5 μl) and OH number. The conversion was >99.5% according to OH number; the output consisted of 26.5% palmitone, 48.6% pentadecyl heptadecyl ketone and 23.4% stearone.

### General Method 2: Preparation of the Secondary Alcohol

### 2.1 Preparation of 18-Pentatriacontanol

130 g of stearone and 10 g of a catalyst (composition 57% CuO, 4% La₂O₃, 24% Al₂O₃, 15% Cu, reduced with hydrogen before use) were introduced into a 0.3 l autoclave and purged three times with nitrogen. The mixture was hydrogenated while stirring (700 rpm at 200° C. and hydrogen pressure 200 bar for 20 hours). After cooling and decompression, the output was dissolved in hot THF, the catalyst was filtered off and the product, after crystallization, was filtered off and dried. The product (120 g) was analyzed with 1H NMR and consisted of 96% 18-pentatriacontanol and 4% stearone.

### General Method 3: Alkoxylation by Means of KOH Catalysis

In a 21 autoclave, the alcohol to be alkoxylated (1.0 eq) is admixed with an aqueous KOH solution which comprises 50% by weight of KOH. The amount of KOH is 0.2% by weight of the product to be prepared. While stirring, the mixture is dewatered at 100° C. and 20 mbar for 2 h. This is followed by purging three times with N₂, establishment of a feed pressure of approx. 1.3 bar of N₂ and a temperature increase to 120 to 130° C. The alkylene oxide is metered in such that the temperature remains between 125° C. and 135° C. (in the case of ethylene oxide) or 130 and 140° C. (in the case of propylene oxide) or 135 and 145° C. (in the case of 1,2-butylene oxide). This is followed by stirring at 125 to 145° C. for a further 5 h, purging with N₂, cooling to 70° C. and emptying of the reactor. The basic crude product is neutralized with the aid of acetic acid. Alternatively, the neutralization can also be effected with commercial magnesium silicates, which are subsequently filtered off. The light-colored product is characterized with the aid of a ¹H NMR spectrum in CDCl₃, gel permeation chromatography and OH number determination, and the yield is determined.

### General Method 4: Alkoxylation by Means of DMC Catalysis (Preferred for C35Sec OH)

In a 21 autoclave, the alcohol to be alkoxylated (1.0 eq) is mixed with a double metal cyanide catalyst (for example DMC catalyst of the Zn—Co type from BASF) at 80° C. To activate the catalyst, approximately 20 mbar is applied at 80° C. for 1 h. The amount of DMC is 0.1% by weight or less of the product to be prepared. This is followed by purging three times with N₂, establishment of a feed pressure of approx. 1.3 bar of N₂ and a temperature increase to 120 to 130° C. The alkylene oxide is metered in such that the temperature remains between 125° C. and 135° C. (in the case of ethylene oxide) or 130 and 140° C. (in the case of propylene oxide) or 135 and 145° C. (in the case of 1,2-butylene oxide). This is followed by stirring at 125 to 145° C. for a further 5 h, purging with N₂, cooling to 70° C. and emptying of the reactor. The light-colored product is characterized with the aid of a ¹H NMR spectrum in CDCl₃, gel permeation chromatography and OH number determination, and the yield is determined.

### General Method 5: Sulfation by Means of Chlorosulfonic Acid

In a 1 l round-bottom flask, the alkyl alkoxylate to be sulfated (1.0 eq) is dissolved in 1.5-times the amount of dichloromethane (based on percent by weight) and cooled to 5 to 10° C. Thereafter, chlorosulfonic acid (1.1 eq) is added dropwise such that the temperature does not exceed 10° C. The mixture is allowed to warm up to room temperature and is stirred under an N₂ stream at this temperature for 4 h before the above reaction mixture is added dropwise to an aqueous NaOH solution of half the volume at max. 15° C. The amount of NaOH is calculated to give rise to a slight excess based on the chlorosulfonic acid used. The resulting pH is approx. pH 9 to 10. The dichloromethane is removed at max. 50° C. on a rotary evaporator under gentle vacuum.

The product is characterized by ¹H NMR and the water content of the solution is determined (approx. 70%).

For the synthesis, the following fatty acids and alcohols were used.

| Fatty acid or | |
|---|---|
| alcohol | Description |
| Stearic acid | C₁₇H₃₅CO₂H |
| C32 Guerbet | Commercially available C₃₂ Guerbet alcohol (2- |
| | tetradecyloctadecan-1-ol), purity >98% |
| C16 | Commercially available fatty alcohol consisting of linear |
| | C₁₆H₃₃—OH |
| C16C18 | Commercially available fatty alcohol consisting of linear |
| | C₁₆H₃₃—OH and linear C₁₈H₃₇—OH |
| C36 Guerbet | Mixture of 80 mol % C₃₆ Guerbet alcohol |
| (80%) | (2-hexadecyleicosan-1-ol) and |
| | 20 mol % octadecanol |

### Performance Tests

The surfactants obtained were used to carry out the following tests in order to assess the suitability thereof for tertiary mineral oil production.

### Description of the Test Methods

### Determination of SP*

### a) Principle of the Measurement:

The interfacial tension between water and oil was determined in a known manner via the measurement of the solubilization parameter SP*. The determination of the interfacial tension via the determination of the solubilization parameter SP* is a method for approximate determination of the interfacial tension which is accepted in the technical field. The solubilization parameter SP* indicates how many ml of oil are dissolved per ml of surfactant used in a microemulsion (Winsor type III). The interfacial tension σ (IFT) can be calculated therefrom via the approximate formula IFT≈0.3/(SP*)², if equal volumes of water and oil are used (C. Huh, J. Coll. Interf. Sc., Vol. 71, No. 2 (1979)).

### b) Procedure

To determine the SP*, a 100 ml measuring cylinder with a magnetic stirrer bar is filled with 20 ml of oil and 20 ml of water. To this are added the concentrations of the particular surfactants. Subsequently, the temperature is increased stepwise from 20 to 90° C., and the temperature window in which a microemulsion forms is observed.

The formation of the microemulsion can be assessed visually or else with the aid of conductivity measurements. A triphasic system forms (upper oil phase, middle microemulsion phase, lower water phase). When the upper and lower phase are of equal size and do not change over a period of 12 h, the optimal temperature (Tₒₚₜ) of the microemulsion has been found. The volume of the middle phase is determined. The volume of surfactant added is subtracted from this volume. The value obtained is then divided by two. This volume is then divided by the volume of surfactant added. The result is noted as SP*.

The type of oil and water used to determine SP* is determined according to the system to be examined. It is possible either to use mineral oil itself or a model oil, for example decane. The water used may either be pure water or saline water, in order better to model the conditions in the mineral oil formation. The composition of the aqueous phase can be adjusted, for example, according to the composition of a particular deposit water.

Information regarding the aqueous phase used and the oil phase can be found below in the specific description of the tests.

### Test Results

A 1:1 mixture of decane and of an NaCl solution was admixed with butyl diethylene glycol (BDG). Butyl diethylene glycol (BDG) functions as a co-solvent and is not included in the calculation of SP*. To this was added a surfactant mixture composed of 3 parts alkyl alkoxysulfate and 1 part dodecylbenzene sulfonate (Lutensit A-LBN 50 ex BASF). The total surfactant concentration is reported in percent by weight of the total volume.

The results are shown in table 1.

TABLE 1
Surfactants on decane

| | Alkyl - AO - SO₄Na: | Surfactant | BDG | NaCl | Tₒₚₜ | | IFT |
|---|---|---|---|---|---|---|---|
| Ex. | C₁₂H₂₅Ph—SO₃Na = 3:1 | [%] | [%] | [%] | [° C.] | SP* | [mN/m] |
| C1 | C16C18 - 9 PO - sulfate | 1.25 | 2 | 4 | 67.6 | 13 | 0.0018 |
| C2 | C32 Guerbet (80%) - 7 | 0.4 | 2 | 5.07 | 71 | 18.25 | 0.0009 |
| | PO - 10 EO - sulfate | | | | | | |
| C3 | C32 Guerbet (90%) - 7 | 0.4 | 2 | 5 | 72 | 26.8 | 0.0004 |
| | PO - 10 EO - sulfate | | | | | | |
| C4 | C32 Guerbet (100%) - 7 | 0.4 | 2 | 4.3 | 72 | 37 | 0.0002 |
| | PO - 10 EO - sulfate | | | | | | |
| C5 | C36 Guerbet (80%) - 7 | 0.8 | 2 | 5.85 | 71 | 7.3 | 0.0056 |
| | PO - 10 EO - sulfate | | | | | | |
| 6 | C35 sec (96%) - 7 PO - | 0.4 | 2 | 3.5 | 71 | 27.6 | 0.0004 |
| | 10 EO - sulfate | | | | | | |
| 7 | C35 sec (96%) - 7 PO - | 0.4 | 2 | 6 | 31 | 54 | 0.0001 |
| | 10 EO - sulfate | | | | | | |
| 8 | C35 sec (96%) - 7 BuO - | 0.4 | 2 | 1.5 | 69 | 51 | 0.0001 |
| | 7 PO - sulfate | | | | | | |

As can be seen in Table 1 in comparative example C1, a standard system based on C16C18—9 PO—sulfate gives an interfacial tension of 0.0018 mN/m on decane. The advantage of this system is the good availability of the surfactant, since the parent C16C18 fatty alcohol is available in a large amount (approx. 200 000 to/y). It is known from the specialist literature (e.g. T. Sottmann, R. Strey “Microemulsions”, Fundamentals of Interface and Colloid Science 2005, Volume V, chapter 5) that the interfacial tension rises with the chain length of the oil used. In order to obtain low interfacial tensions on heavy oils, a surfactant with a relatively long hydrophobic moiety is therefore needed. Remaining with the model oil decane, surfactant systems with interfacial tensions of <0.001 mN/m are consequently of great interest.

The structure of such surfactants requires alcohols which should have 30 or more carbon atoms. Linear or lightly branched alcohols in this carbon chain range (e.g. Ziegler alcohols by ethylene oligomerization and subsequent introduction of the alcohol group) are available only in extremely small amounts and are not an option for tertiary mineral oil production.

The only alcohols known to date on the market are long-chain Guerbet alcohols. These are prepared by dimerizing alcohols with elimination of water, and are primary alcohols with a branch in the 2 position. However, the longer the alcohol used, the more difficult this dimerization is, i.e. the conversion rates are incomplete (in the case of Guerbet alcohols having more than 28 carbon atoms they are usually only 70%). Therefore, long-chain Guerbet alcohols had good industrial availability only as a mixture of the Guerbet alcohol and the low molecular weight alcohol which was used as the starting material. A C32 Guerbet (80%) is therefore a mixture of 80% C32 Guerbet alcohol and 20% C16 alcohol. As can be seen with reference to comparative examples C2, C3 and C5, the interfacial tension is only below 10⁻³ mN/m if very pure Guerbet alcohols (>>80%) can be prepared.

If, however, pure Guerbet alcohols which have more than 30 carbon atoms are desired, this requires distillation to remove the low molecular alcohol. This complicates the production and makes it more expensive. C4 shows a specimen based on a pure and hence expensive Guerbet alcohol. It is possible here to achieve extremely low interfacial tensions.

Surfactants claimed (examples 6-8) give interfacial tensions of 10⁻⁴ mN/m. They are technically simpler to prepare than surfactants based on corresponding long-chain pure Guerbet alcohols. Instead of hydrogenating fatty acids having 16 or more carbon atoms to fatty alcohols and then dimerizing them incompletely in the Guerbet reaction and distilling off the rest of the fatty alcohol, it is possible to directly join the fatty acid to the corresponding ketone and finally to reduce it to the alcohol. The conversion rates are virtually quantitative.

1.-14. (canceled)

15. A surfactant of the general formula (I)
(R¹)(R²)—CH—O-(D)ₙ-(B)ₘ-(A)ₗ-XY^{a-}a/bM^{b+}, (I)
wherein
R¹ is a linear or branched, saturated or unsaturated, aliphatic hydrocarbon radical having 7 to 21 carbon atoms,
R² is a linear or branched, saturated or unsaturated, aliphatic hydrocarbon radical having 7 to 21 carbon atoms, and
R¹ is either identical to R² or has a maximum of four more carbon atoms than R²,
A is ethyleneoxy,
B is propyleneoxy, and
D is butyleneoxy,
l is from 0 to 99,
m is from 0 to 99 and
n is from 0 to 99,
X is an alkyl or alkylene group having 0 to 10 carbon atoms,
M^{b+} is a cation,
Y^{a-} is selected from the group of sulfate groups, sulfonate groups, carboxylate groups and phosphate groups,
b is 1, 2 or 3, and
a is 1 or 2, where
the A, B and D groups may be distributed randomly, alternatingly, or in the form of two, three, four or more blocks in any sequence, the sum of l+m+n is in the range from 0 to 99 and the proportion of 1,2-butylene oxide, based on the total amount of butylene oxide, is at least 80%.

16. The surfactant according to claim 1, wherein the sum of l+m+n is in the range from 3 to 50.

17. The surfactant according to claim 1, wherein the proportion of 1,2-butylene oxide, based on the total amount of butylene oxide, is at least 90%.

18. The surfactant according to claim 15, wherein the surfactant comprises 2 to 15 1,2-butylene oxide units.

19. The surfactant according to claim 15, wherein R¹ is either identical to R² or has at most two more carbon atoms than R² and is a linear or branched, saturated or unsaturated, aliphatic hydrocarbon radical having 15 to 17 carbon atoms.

20. The surfactant according to claim 15, wherein R¹ is identical to R² and is a linear, saturated or unsaturated, aliphatic hydrocarbon radical having 17 carbon atoms.

21. The surfactant according to claim 15, wherein
m is from 5 to 9 and
n is from 2 to 10, and
Y⁻ is selected from the group of sulfate groups, sulfonate groups, and carboxylate groups, where
the A, B and D groups are present to an extent of more than 80% in block form in the sequence D,B,A, beginning from (R¹)(R²)—CH—, the sum of l+m+n is in the range from 7 to 49, and the proportion of 1,2-butylene oxide, based on the total amount of butylene oxide in the molecule, is at least 90%.

22. The surfactant according to claim 20, wherein
m is from 5 to 9 and
n is from 2 to 10, and
Y⁻ is selected from the group of sulfate groups, sulfonate groups, and carboxylate groups, where
the A, B and D groups are present to an extent of more than 80% in block form in the sequence D,B,A, beginning from (R¹)(R²)—CH—, the sum of l+m+n is in the range from 7 to 49, and the proportion of 1,2-butylene oxide, based on the total amount of butylene oxide in the molecule, is at least 90%.

23. The surfactant according to claim 22, wherein the surfactant comprises 2 to 15 1,2-butylene oxide units.

24. A process for mineral oil extraction which comprises Winsor type III microemulsion flooding, in which an aqueous surfactant formulation comprising at least one ionic surfactant, for the purpose of lowering the interfacial tension between oil and water to <0.1 mN/m, is injected through at least one injection borehole into a mineral oil deposit, and crude oil is withdrawn from the deposit through at least one production borehole, wherein the surfactant formulation comprises at least one surfactant as claimed in claim 15.

25. The process according to claim 24, wherein the concentration of all surfactants together is 0.05 to 5% by weight, based on the total amount of the aqueous surfactant formulation.

26. A surfactant formulation comprising at least one ionic surfactant as claimed in claim 15.

27. The surfactant formulation according to claim 26, wherein the concentration of all surfactants together is 0.05 to 5% by weight, based on the total amount of the aqueous surfactant formulation.

28. A process for preparing a surfactant according to claim 15, comprising:
(a) preparing ketones of the general formula (II) 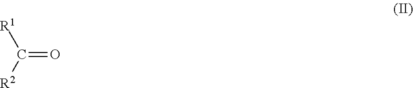
wherein
R¹ is a linear or branched, saturated or unsaturated, aliphatic hydrocarbon radical having 7 to 21 carbon atoms,
R² is a linear or branched, saturated or unsaturated, aliphatic hydrocarbon radical having 7 to 21 carbon atoms, and
R¹ is either identical to R² or has a maximum of four more carbon atoms than R², by reacting two carboxylic acids of the general formulas IIa and IIb,
R¹—COOH (IIa)
R²—COOH (IIb),
in each of which R¹ and R² are defined above,
(b) hydrogenating the ketone of the general formula (II) prepared in process step (a) to give the secondary alcohol of the formula R¹(R²)—CHOH,
(c) alkoxylating the secondary alcohols of the formula R¹(R²)—CHOH obtained in process step (b), and
(d) introducing the anionic group XY⁻.

29. The process according to claim 28, in which step (a) is performed in the gas phase in the presence of a catalyst at 300-600° C.

30. The process according to claim 28, in which step (b) is performed continuously or batchwise at a temperature of 100 to 320° C. and a pressure of 100 to 325 bar.

31. The process according to claim 29, in which step (b) is performed continuously or batchwise at a temperature of 100 to 320° C. and a pressure of 100 to 325 bar.
